(19) United States
(12) Patent Application Publication
(10) Pub. No.: US 2011/0280848 A1
(43) Pub. Date: Nov. 17, 2011

(54) COMPOSITION COMPRISING CHICORIC ACID AND/OR DERIVATIVES THEREOF

(75) Inventors: Rachid Bel-Rhlid, Savigny (CH); Vanessa Crespy, Dublin, OH (US); Thomas Wolfgang Raab, Grandvaux (CH); Nicole Page-Zoerkler, Epalinges (CH); Rene Fumeaux, Blonay (CH); Maricel Marin-Kuan, Lausanne (CH); Dominique Piguet, Pompaples (CH)

(73) Assignee: NESTEC S.A., Vevey (CH)

(21) Appl. No.: 13/145,656

(22) PCT Filed: Jan. 27, 2010
(86) PCT No.: PCT/EP2010/050914

§ 371 Date: Jul. 21, 2011

(30) Foreign Application Priority Data
Jan. 27, 2009 (EP) 09151390.3

(51) Int. Cl. A61K 35/74; C12P 7/42; A61P 19/08; A61P 37/02; A61P 39/06; A61P 3/10; C12P 7/46; A61P 29/00

(52) U.S. Cl. 424/93.45; 424/93.4; 435/145; 435/146

(57) ABSTRACT

The present invention relates generally to the field of food and drinks. In particular, for example, a composition is provided that allows to provide tartaric and/or caffeic acid to a subject. One embodiment of the present invention is a composition comprising an ingredient containing chicoric acid and/or derivatives thereof, and a lactic acid bacterium capable of hydrolysing chicoric acid and/or derivatives thereof to generate tartaric and/or caffeic acid.

The present invention relates generally to the field of food and drinks. In particular, for example, a composition is provided that allows to provide tartaric and/or caffeic acid to a subject. One embodiment of the present invention is a composition comprising an ingredient containing chicoric acid and/or derivatives thereof, and a lactic acid bacterium capable of hydrolysing chicoric acid and/or derivatives thereof to generate tartaric and/or caffeic acid.

Caffeic Acid (CA) was shown to have tumor-shrinking properties. The subcutaneous and oral administrations of CA significantly reduced liver metastasis. These results confirm the therapeutic potential of CA and suggest that the anti-metastatic and anti-tumor effects of CA are mediated through the selective suppression of MMP-9 enzyme activity and transcriptional down-regulation by the dual inhibition of NF-κB as well as MMP-9 catalytic activity. [Tae-Wook Chung, et al., FASEB Journal. 2004; 18:1670-1681]

The caffeic acid derivative, caffeic acid phenethyl ester (CAPE), is known to have antimitogenic, anticarcinogenic, anti-inflammatory, and immunomodulatory properties. [Natarajan et al., Proc Natl Acad Sci USA. 1996 Aug. 20; 93(17): 9090-9095] CAPE also suppresses acute immune and inflammatory responses [Orban et al., Neurolmmuno Modulation 2000; 7:99-105]

The anti-inflammatory and anti-cancer property has also been shown to protect skin cells when exposed to ultraviolet (UV) radiation, in particular UVC radiation [NERADIL, R. et al., Folia Biologica (Praha). 2003; 49:197-202] and UVB radiation. [Staniforth et al., Carcinogenesis 2006 27(9):1803-1811].

Tartaric acid is known to provide the following health benefits: It can increase the stool softeners, decrease intestinal transit time (Spiller et al., British Journal of Nutrition (2003), 90, 803-807) and may participate in the acid base status (Sabboh et al., (2007), 98, 72-77.

Consequently, it would be desirable to have available food product with caffeic acid and/or tartaric acid to produce the benefits described above. However, simply supplementing a foodstuff with caffeic acid and/or tartaric acid would not be ideal, since these compounds might loose activity with time.

It was hence the object of the present invention to prepare a foodstuff that can provide a subject with caffeic acid/and or tartaric acid that is storage stable and easy to produce.

The present inventors could achieve this object by providing a food composition that allows to produce caffeic acid/and or tartaric acid in situ from a precursor, chicoric acid and/or derivatives thereof.

Chicoric acid is

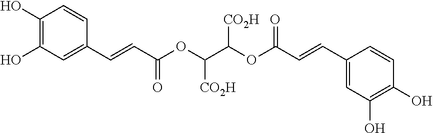

and derivatives of chicoric acid include

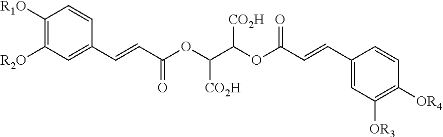

R₁, R₂, R₃ and/or R₄ may be identical or may differ from one another.

In one embodiment, R₁, R₂, R₃ and/or R₄ may be selected from the group consisting of H; CH₃; C₁-C₃-alkyl; aryl, such as phenyl, benzyl, tolyl, o-xylylalkyl; C₁-C₃-acyl; amino acids; mono-, di- or oligosaccharides. Oligosaccharides contain between two and nine monosaccharide units. R₁, R₂, R₃ and R₄ may be identical and/or may differ from one another.

One typical derivative of chicoric acid is for example the following compound:

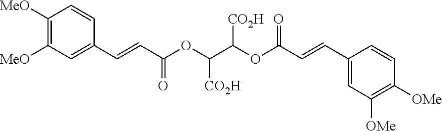

Chicoric acid and/or its derivatives can then be hydrolysed by a lactic acid bacterium capable of hydrolysing chicoric acid and/or derivatives thereof. This hydrolysis step will generate tartaric and/or caffeic acid.

Without wishing to be bound by theory, the inventors believe that this hydrolysis occurs as outlined in the following scheme. Note, that all reaction steps catalyzed by “enzyme” may also be catalyzed by the lactic acid bacterium described in the present application and/or by combinations of lactic acid bacterium and enzymes.

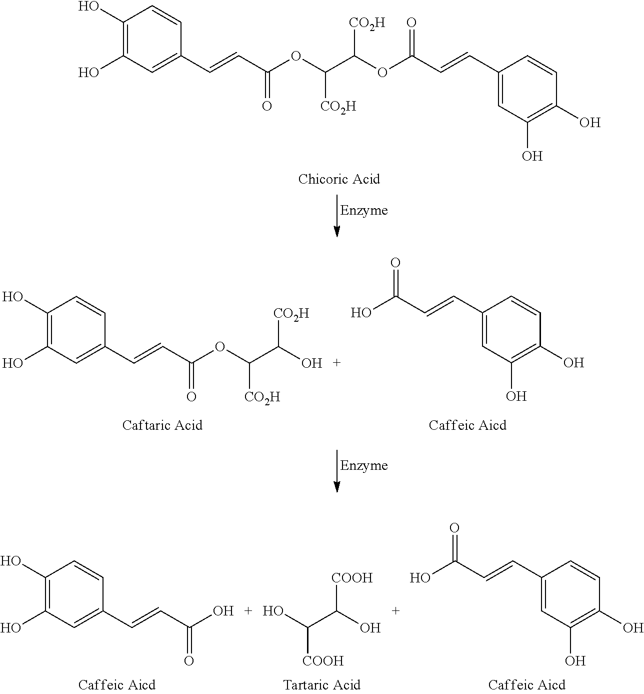

The inventors have surprisingly found that treating an ingredient comprising chicoric acid and/or derivatives thereof with lactic acid bacterium capable of hydrolysing chicoric acid and/or derivatives thereof to generate tartaric and/or caffeic acid results for example in improved antioxidant and/or anti-inflammatory properties of the ingredient.

Furthermore, it has been found that this treatment can take place in vivo when a human or an animal ingests a composition comprising chicoric acid and/or derivatives thereof in combination with a lactic acid bacterium capable of hydrolysing chlorogenic acids to generate phenolic acids.

Accordingly one embodiment of the present invention is a composition comprising an ingredient containing chicoric acid and/or derivatives thereof, and a lactic acid bacterium capable of hydrolysing chicoric acid and/or derivatives thereof to generate tartaric and/or caffeic acid.

The composition of the present invention may be intended for oral administration, preferably as food product, food supplement or drink, or for parenteral application.

In a preferred embodiment the ingredient is enriched in chicoric acid and/or derivatives thereof. For example, the ingredient and/or the composition may comprise chicoric acid and/or derivatives thereof in an amount in the range of 0.001-99.99 weight-% of dry weight, preferably 0, 1-50 weight-%) of dry weight, most preferred 0.1-10 weight-% of dry weight. The ingredient and/or the composition may comprise the lactic acid bacterium capable of hydrolysing chicoric acid and/or derivatives thereof to generate tartaric and/or caffeic acid in an amount in the range of 0.001-99.99 weight-% of dry weight, preferably 0.1-50 weight-% of dry weight, most preferred 0, 1-10 weight-% of dry weight.

For example the composition of the present invention may comprise an ingredient containing chicoric acid and/or derivatives thereof and another ingredient comprising a lactic acid bacterium capable of hydrolysing chicoric acid and/or derivatives thereof to generate tartaric and/or caffeic acid.

The ingredient containing chicoric acid and/or derivatives thereof may be any ingredient that contains chicoric acid and/or derivatives thereof, either naturally or in added form, but is preferably a natural foodstuff such as lettuce, chicory, dandelion, grape, grape pomace; or combinations or extracts thereof.

The ingredient to be mixed with the ingredient containing chicoric acid and/or derivatives thereof comprises a lactic acid bacterium capable of hydrolysing chicoric acid and/or derivatives thereof to generate tartaric and/or caffeic acid. These two ingredients may be mixed briefly prior to consumption or may be provided as a ready-to-consume composition.

Preferred lactic acid bacterium capable of hydrolysing chicoric acid and/or derivatives thereof to generate tartaric and/or caffeic acid are probiotic lactic acid bacterium having a esterase activity, such as chlorogenate esterase and/or feruloyl esterase, preferably Lactobacillus or Bifidobacterium, for example L. johnsonii, B. longum, and B. lactis (CNCM I-3446)., even more preferred Lactobacillus johnsonii La1 (CNCM I-1225), B. longum BB 536, and B. lactis BB12.

B. longum BB 536 is commercially available from Morinaga Nutritional Foods, Inc.

B. lactis BB12 is commercially available, e.g., from Chr. Hansen, DK-2970 Horsholm.

In one embodiment of the present invention, the lactic acid bacterium may be used in a non-replicating form.

The ability of a lactic acid bacterium or of a fraction thereof to hydrolyse chicoric acid and/or derivatives thereof to generate tartaric and/or caffeic acid may be tested as described in detail for L. johnsonii (La1) in Examples 2, 3 and/or 4.

The lactic acid bacterium should be present in an amount sufficient for hydrolysing a substantial amount of chicoric acid to generate tartaric and/or caffeic acid during digestion. The amount of lactic acid bacterium and/or enzyme needed may e.g. be determined by those skilled in the art, for example dependent on the subject to be treated or on the speed by which the tartaric and/or caffeic acid should be liberated. Preferably at least 5%, such as at least 30%, at least 50%, or at least 75% of chicoric acid present in the composition is hydrolysed prior to and/or during consumption.

In another embodiment, an enzyme capable of hydrolysing chicoric acid to generate tartaric and/or caffeic acid is further added to the lactic acid bacterium. Preferably, such enzyme is selected from the group consisting of esterases, such as chlorogenate esterase, tannase and/or feruloyl esterase. It may be added in an amount such as preferably at least 5%, such as at least 30%, at least 50%, or at least 75% of chicoric acid present in the composition is hydrolysed prior to and/or during consumption.

Suitable enzymes that can be used in the framework of the present invention include e.g. esterases, e.g. a chlorogenate esterase derived from Aspergillus japonicus. (Commercially available from Kikkoman, Japan), Tannase from Aspergillus oryzae (EC 3.1.1.20) (commercially available from Kikkoman, Japan); and Palatase 20000L (EC 3.1.1.3) (commercially available from Novozymes A/S, Denmark). The enzyme may be present as a purified enzyme or e.g. in the form of a cell lysate of a microorganism. Suitable cells may e.g. be cells of the microroganisms mentioned above. Suitable methods for producing cell lysate are known in the art.

The composition and/or ingredients of the invention should be formulated such that the lactic acid bacterium strain will not ferment or react with the composition during storage. This may be achieved e.g. by formulating the composition as a dry powder, and/or by encapsulating the lactic acid bacterium so that it will only be released when the composition is mixed with at least one other ingredient or during digestion.

The composition of the present invention may be a nutritional complete formula, a dairy product, a chilled or shelf stable beverage, a product for lactating mothers, a liquid drink, a soup, a dietary supplement, a meal replacement, a nutritional bar, a confectionery, a milk or a fermented milk product, a yoghurt, a milk based powder, an enteral nutrition product, an infant formula, an infant nutritional product, a puree, a cereal product or a fermented cereal based product, an ice-cream, candy, sweets, biscuits, cakes, a chocolate, a cappuccino, a coffee, a culinary product such as mayonnaise, tomato puree or salad dressings, a pet food or a pet beverage.

If the product is a nutritional supplement for oral administration it may be present in capsules, gelatin capsules, soft capsules, tablets, sugar-coated tablets, pills, pastes or pastilles, gums, or drinkable solutions or emulsions, a syrup or a gel. Such a supplement might also include a sweetener, a stabilizer, an antioxidant, an additive, a flavouring agent and/or a colorant.

The individual ingredients may comprise any kind of edible compound. Typical ingredients for food compositions, in particular drinks, are well known in the art, e.g. milk, cream, coffee whiteners, and coffee creamers. Alternatively, ingredients could also be a salad dressing or parts thereof, for example. Such compounds are used by consumers to modify e.g. the aroma, appearance and texture of a composition. The ingredients may be in liquid or dry form, e.g. as powders, that are dissolved and/or suspended in a drink.

The composition of the invention may further comprise further ingredient suitable for inclusion in a food composition. Usual ingredients may e.g. be sugars, artificial sweeteners, emulsifiers, stabilisers, thickeners, flowing agents, colours, flavours, aromas, and the like. Suitable artificial sweeteners include saccharin, cyclamates, acetosulfame, L-aspartyl based sweeteners such as aspartame, and mixtures of these. Suitable emulsifiers include monoglycerides, diglycerides, lecithin, diacetyl tartaric acid esters of mono-diglycerides, emulsifying starches, and mixtures thereof. Suitable stabilisers include dipotassium phosphate and sodium citrate. A suitable flowing agent is sodium silica aluminate. In one embodiment the composition comprises milk protein and/or vegetable protein. In a further embodiment the composition comprises milk fat and/or vegetable fat.

The composition of the present invention may comprise a protein source, a carbohydrate source and/or a lipid source. If the composition is intended to be used as a full meal or as a meal replacement it comprises preferably a protein source, a carbohydrate source and a lipid source, so that the nutritional requirements of the subject to be treated are satisfied.

The protein source for example may comprise milk protein and/or vegetable protein, the lipid source may comprise milk fat and/or vegetable fat, and/or the carbohydrate source may comprise milk and/or vegetable carbohydrates, to ensure an optimal nutritional value.

Further, the composition may contain an organic or inorganic carrier material suitable for oral or enteral administration as well as vitamins, minerals trace elements and other micronutrients, for example in accordance with the recommendations of Government bodies such as the USRDA.

In one embodiment the invention relates to a beverage powder comprising: a) a dried water-soluble composition comprising chicoric acid and/or derivatives extract; and b) a lactic acid bacterium capable of hydrolysing chicoric acid and/or derivatives thereof to generate tartaric and/or caffeic acid.

A beverage powder according to the invention is a powder to be used for the preparation of a beverage by dissolving or suspending the powder in a liquid, e.g. water or milk.

The products of the invention may be used to enhance antioxidant capacity in vivo in a human or animal consuming, e.g., a beverage prepared from the products of the invention, e.g. by increasing the Nrf2-mediated gene expression pathway i.e inducing detoxifying enzymes such as gluthathione-S-transferase (GST). An increased activity of Nrf2 associated genes has been reported to enhance detoxification and to stimulate the endogenous defense against oxidative stress.

The composition of the invention may be used for example to decrease inflammation, e.g. by reducing the prostaglandin E2 level.

Many health problems and disorders, in particular age related disorders, are related to oxidative stress and inflammation. The products of the invention may be used to treat or prevent such problems or disorders in a human or animal consuming a beverage prepared from the products of the invention. Relevant problems and disorder are e.g brain disorders; inflammation; obesity; and cancer.

The composition of the present invention may further be used as anti-diabetic agents, e.g. by reducing blood glucose levels, and/or increasing blood levels of leptin, insulin and/or c-peptide; as bone remodeling agents, e.g. by increasing bone mineral density, e.g. by increasing serum levels of estrogen and/or progesterone and/or alkaline phosphatase activity; as anti-metastatic agents, e.g. with anti-angiogenic effect.

Consequently, one embodiment of the present invention is the use of a composition as described herein for the preparation of a composition to treat or prevent inflammatory disorders, in particular linked to bacterial and/or viral infection; to enhance antioxidant capacity in a human or animal; to treat or prevent oxidative stress related disorders; to treat or prevent metabolic disorders, such as diabetes; to treat or prevent disorders related to bone mineral density; or to treat or prevent cancer.

In one embodiment the invention relates to a kit for preparing a food product, for example a beverage, comprising at least two parts: a) a first part comprising a chicoric acid and/or derivatives thereof containing food ingredient; and b) a second part comprising a lactic acid bacterium capable of hydrolysing chicoric acid and/or derivatives thereof to generate tartaric and/or caffeic acid. The two parts may be sold together for the preparation of a food product, for example a beverage, but may be physically separated in the packing of the product. The final food product to be consumed may be prepared by mixing the two parts shortly before consumption. If one or both parts are in a liquid form they may be mixed directly, optionally further liquid, e.g. water or milk, may be added. The two parts may also be mixed by dissolving or suspending them in a liquid, e.g. water or milk. When liquid is used this may be hot or cold. If hot liquid is used, it may preferably have a temperature which is not so high as to inactivate the lactic acid bacterium and enzyme if any, before ingestion of the foodstuff, food supplement or beverage.

The first part of the kit may comprise a food product or an extract containing chicoric acid and/or derivatives thereof. In a preferred embodiment the first part is in a dry form, e.g. in the form of a powder. The first part may also be in a liquid form. The first part may additionally comprise any other suitable ingredient, e.g., aroma additives, stabilisers, salts, and/or sweeteners. The first part may be packed in any suitable way, e.g. in a sachet, bottle or can.

The second part comprises a lactic acid bacterium capable of hydrolysing chicoric acid and/or derivatives thereof to generate tartaric and/or caffeic acid. This part may preferably be in the form of a composition to be mixed with the first part. It may be in dry form, e.g. as a powder, or in liquid form, and may be packed in any suitable way, e.g. in a sachet, bottle or can. It should be formulated such that the lactic acid bacterium will not ferment or react with other ingredients during storage. This may be achieved e.g. by formulating the composition as a dry powder, and/or by encapsulating the lactic acid bacterium.

The at least two parts may be packed together in any suitable way. They may e.g. be packed in a combined container wherein the parts are kept physically separated during storage and mixed when the container is opened, or they may be packed in separate containers which are sold together for the preparation of a foodstuff, food supplement or beverage.

Those skilled in the art will understand that they can freely combine all features of the present invention described herein, without departing from the scope of the invention as disclosed. In particular, features described for the uses of the present invention may be applied to the composition and/or the kit of the present invention and vice versa.

Further advantages and features of the present invention are apparent from the following Examples and Figures.

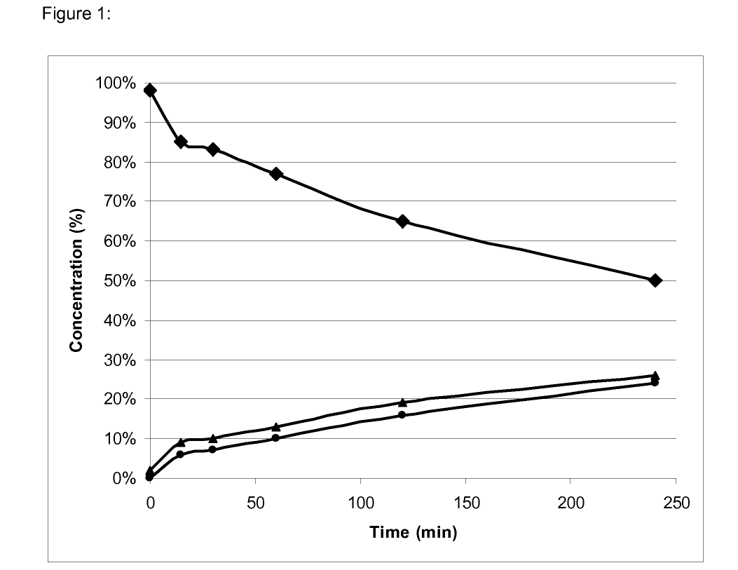

FIG. 1 shows the hydrolysis of chicoric acid (♦) with chlorogenate esterase into caffeic acid (▴) and caftaric acid ().

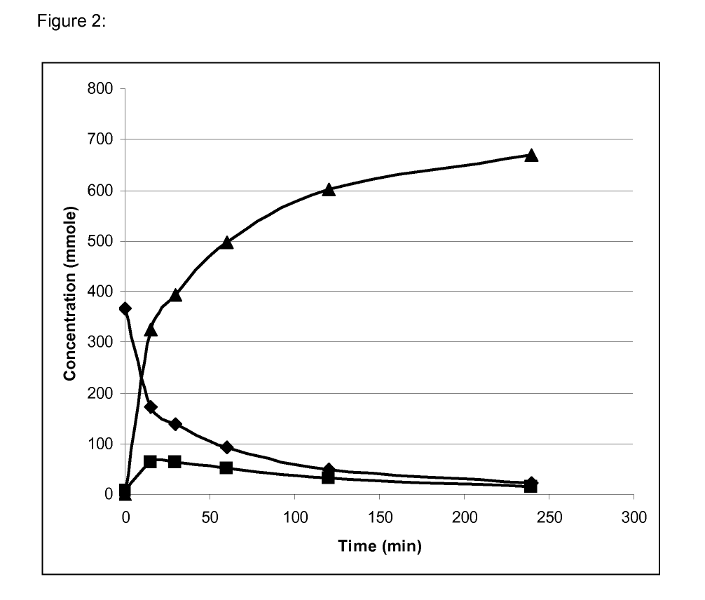

FIG. 2 shows the hydrolysis of chicoric acid (♦) with a spray-dried preparation of L. johnsonii into caffeic acid (▴) and caftaric acid (▪).

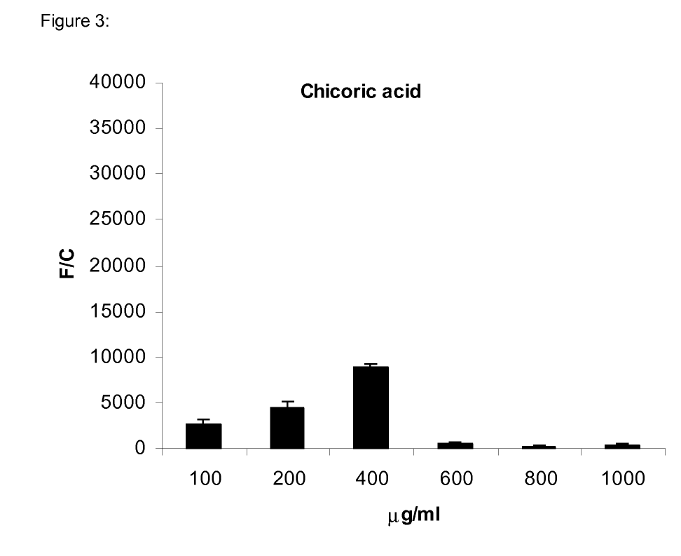

FIG. 3 shows the results of a AREc reporter cell line treated with chicoric acid.

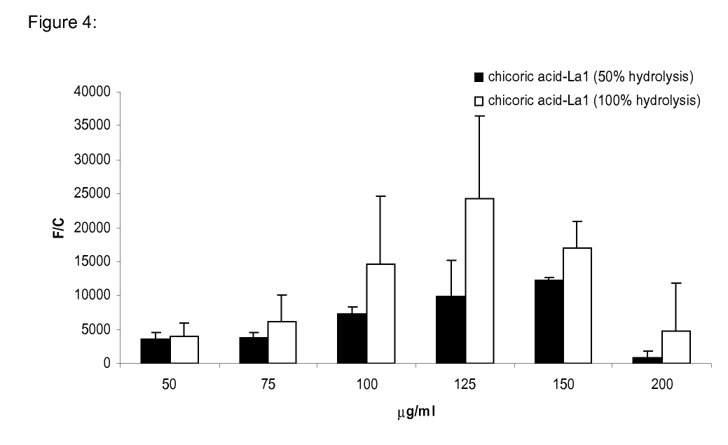

FIG. 4 shows the results of a AREc reporter cell line treated with chicoric acid hydrolysed by La1: (▪) 50% hydrolysis, (□) 100% hydrolysis

### EXAMPLES

### 1—Hydrolysis of Chicoric Acid with Chlorogenate Esterase

A solution of chlorogenate esterase (0.8 mg, 24 U/g, from Kikkoman Japan) in 100 μl phosphate buffer (50 mM, pH 7.0) was added to a solution of chicoric acid (0.57 mg) in 100 μl phosphate buffer (50 mM, pH 7.0). The mixture was then incubated at 37° C. for 4 h. After the reaction time, the enzymatic activity was stopped by heat treatment (5 min, 90° C.) and the mixture was centrifuged (microcon YM10, 30 min, 14000 g). The supernatant was then analysed by HPLC. A control reaction was run in parallel under the same reaction conditions but without enzyme.

### 2—Hydrolysis of Chicoric Acid with L. johnsonii (La1) Fresh Cells

Cells of L. johnsonii (CNCM 1-1225) were grown (7.0 E08 cfu/ml) and centrifuged (5000 g, 10 min), the pellets were resuspended in phosphate buffer (50 mM, pH 7.0) at a concentration of 0.61 g/ml. To 100 μl of this cells solution, 100 μl of a solution of chicoric acid (12 mM) was added and the mixture was incubated at 37° C. Samples were withdrawn at different reaction times, centrifuged (3000 g, 5 min), filtered through 0.45 μm pore size syringe filters (Millipore SLHA 025 BS) and analysed by HPLC.

A control reaction was run in parallel under the same reaction conditions but without bacterium.

### 3—Hydrolysis of Chicoric Acid with L. johnsonii Extract (Lysed Cells)

Cells of L. johnsonii (CNCM 1-1225) were grown (7.0 E08 cfu/ml) and centrifuged (5000 g, 10 min), the pellets were resuspended in phosphate buffer (50 mM, pH 7.0) at a concentration of 0.61 g/ml. The cells were then lysed using the glass-beads method. 600 μl of cells preparation were put into a Mini-Beadbeater for 1 min of intense shaking, cooled in ice, and put another 1 min in the Mini-Beadbeater. The crude cell extract (100 μl) was then added to 100 μl of a solution of chicoric acid (12 mM, phosphate buffer 50 mM, pH 7.0) and the mixture was incubated at 37° C. Samples were withdrawn at different reaction times, centrifuged (3000 g, 5 min), filtered through 0.45 μm pore size syringe filters (Millipore SLHA 025 BS) and analysed by HPLC.

### 4—Hydrolysis of Chicoric Acid with a Spray-Dried Preparation of La1

10 mg of a spray-dried preparation of La1 (3.3 E09 cfu/g) were dissolved in 100 μl of phosphate buffer (50 mM, pH 7.0). To this solution, 100 μl of a chicoric acid solution (12 mM, phosphate buffer 50 mM, pH 7.0) were added. The mixture was then incubated at 37° C. and samples were withdrawn at different reaction times. After centrifugation (3000 g, 5 min) and filtration (0.45 μm pore size syringe filters, Millipore SLHA 025 BS) the samples were analysed by HPLC.

### HPLC Analysis

HPLC-DAD analysis of chicoric acid and hydrolysis products was performed on Agilent 1100 system equipped with Atlantis C18 reverse-phase column (4.6×100 mm, particle size 3 μm) and a diode array detector. The column was equilibrated with water containing 0.1% formic acid. After injection, a linear gradient to a final solvent composition of 55% water and 45% acetonitrile (containing 0.1% formic acid) was run within 12 min at a flow rate of 1 ml/min. Chicoric acid and caffeic acid were monitored by UV at 320 nm and were quantified using standard calibration curves.

### 5—In Vitro Tests

### Antioxidant Responsive Element (ARE) Luciferase Assay

The pGL-8×ARE which contains eight copies of the ARE present in rat glutathione-S-transferase A2 (GSTA2) along with the pcDNA3.1 plasmid containing the neomycin selectable marker was stably transfected into human MCF7 cells (Wang et al., Cancer Res. 66, 10983-10994, 2006). ARE (antioxidant-responsive element) is the binding site of the transcription factor Nrf2 which regulates the genes involved in detoxification and endogenous defense against oxidative stress. The plasmid pGL-8×ARE contains a luciferase gene downstream of the eight Nrf2 binding sites that allows monitoring Nrf2 activity. For treatment with chicoric acid and related compounds, the AREc 32 cells were seeded 96-well microtiter plates in DMEM growth medium. After treatment of 24 h with the different preparations, firefly luciferase activity was determined.

### RESULTS

### Tested Bacteria

| Bacteria | Culture Media |
|---|---|
| Lactobacillus rhamnosus GG (NCC 4007) | MRS |
| Lactobacillus johnsonii La1 (CNCM I-1225) | MRS |
| Lactobacillus paracasei ST11 (NCC 2461) | MRS + Cysteine |
| Bifidobacterium longum BB 536 (ATCC BAA-999) | MRS + Cysteine |
| Bifidobacterium lactis BB12 (CNCM I-3446) | MRS |
| Streptococcus thermophilus TH4 (NCC 2496) | HJL |

In particular L. johnsonii (La1), B. longum BB 536, and B. lactis BB12 were able to hydrolyse chicoric acid. The best results in term of reaction rate and reaction yield were obtained with L. johnsonii (La1)

### Tested Enzymes

| Enzyme | Supplier |
|---|---|
| Chlorogenate esterase | Kikkoman, Japan |
| Feruloyl esterase | Novozymes |
| Porcine liver esterase | Sigma E-3019 |
| Hog liver esterase immobilised on Eupergit C | Fluka 46064 |
| Esterase from Saccharomyces cerevisiae | Fluka 46071 |

Only chlorogenate and feruloyl esterases were able to hydrolyse chicoric acid into caffeic and tartaric acids

1—Hydrolysis of chicoric acid with chlorogenate esterase

TABLE 1
Hydrolysis of chicoric acid into caffeic and caftaric acids by
chlorogenate esterase. Concentration in % relative to untreated
reference at t = 0

Time (min)

| | 0 | 15 | 30 | 60 | 120 | 240 |
|---|---|---|---|---|---|---|
| chicoric Acid | 98 | 85 | 83 | 77 | 65 | 50 |
| Caftaric Acid | 0 | 6 | 7 | 10 | 16 | 24 |
| Caffeic Acid | 2 | 9 | 10 | 13 | 19 | 26 |

2—Hydrolysis of Chicoric Acid with La1 Fresh Cells

After 4h reaction time all chicoric acid was transformed into caffeic acid as analysed by HPLC

3—Hydrolysis of Chicoric Acid with La1 Extract (Lysed Cells)

As compared to whole cells, the hydrolysis of chicoric acid with lysed cells resulted in an increase of the reaction rate. Indeed, after only 2h all chicoric acid was transformed into caffeic acid

4—Hydrolysis of Chicoric Acid with a Spray-Dried Preparation of La1

TABLE 2
Hydrolysis of chicoric acid into caffeic and caftaric acids by a
spray-dried preparation of L. johnsonii. Concentration in % relative to
untreated reference at t = 0

| | 0 min | 15 min | 30 min | 1 h | 2 h | 3 h |
|---|---|---|---|---|---|---|
| Chicoric Acid (mmole) | 365 | 172 | 137 | 91 | 49 | 23 |
| Caftaric Acid (mmole) | 0 | 62 | 63 | 50 | 31 | 15 |
| Caffeic Acid (mmole) | 0 | 324 | 393 | 498 | 601 | 669 |

### 5—In-Vitro Tests:

### Antioxidant Responsive Element (ARE) Luciferase Assay

Human breast cancer cells (AREc32) stably transfected with several copies of the rat GSTA2-ARE reporter construct was used to demonstrate the activation of Nrf2-ARE pathway by chicoric acid and related compounds. Chicoric acid not hydrolysed and chicoric acid hydrolysed with La1 (50% and 100%) produced a positive dose-dependent response in Nrf2-luciferase reporter activity (see tables 3, 4), exhibiting a saturation inactivation response at higher doses.

TABLE 3
AREc reporter cell line treated with chicoric acid

| μg/ml | F/C | stdv |
|---|---|---|
| 100 | 2580 | 592 |
| 200 | 4364 | 876 |
| 400 | 8810 | 492 |
| 600 | 558 | 105 |
| 800 | 251 | 166 |
| 1000 | 346 | 214 |

TABLE 4
AREc reporter cell line treated with hydrolysed chicoric acid by
La1: (A) 50% hydrolysis, (B) 100% hydrolysis

| μg/ml | F/C A | F/C B | Stdv-A | Stdv-B |
|---|---|---|---|---|
| 50 | 3584 | 3924 | 934 | 2070 |
| 75 | 3732 | 6157 | 812 | 3811 |
| 100 | 7230 | 14579 | 1141 | 9995 |
| 125 | 9797 | 24309 | 5387 | 12053 |
| 150 | 12208 | 16900 | 332 | 3903 |
| 200 | 764 | 4652 | 987 | 7151 |

1. A composition comprising an ingredient containing chicoric acid and/or derivatives thereof, and a lactic acid bacterium capable of hydrolysing chicoric acid and/or derivatives thereof to generate tartaric and/or caffeic acid.

2. The composition of claim 1, comprising a protein source, a carbohydrate source and a lipid source.

3. The composition of claim 2, wherein the protein source comprises a protein selected from the group consisting of milk protein and vegetable protein, the lipid source comprises a lipid selected from the group consisting of milk fat and vegetable fat, and the carbohydrate source comprises a carbohydrate selected from the group consisting of milk and vegetable carbohydrates.

4. The composition of claim 1 wherein the composition is selected from the group consisting of a food product, a food supplement, a drink, and a composition for parenteral application.

5. The composition of claim wherein the composition is in the form of a powder.

6. The composition claim 1 wherein the lactic acid bacterium is a probiotic lactic acid bacterium having an esterase activity.

7. The composition of claim 1 wherein the lactic acid bacterium is selected from the group consisting of L. johnsonii, B. longum, and B. lactis in particular Lactobacillus johnsonii (CNCM 1-1225), B. longum BB 536, B. lactis (CNCM 1-3446) and B. lactis BB12.

8. The composition of claim 1, wherein the chicoric acid containing ingredient is a natural foodstuff.

9. The composition of claim 1, comprising an enzyme capable of hydrolysing chicoric acid to generate tartaric and/or caffeic acid.

10. A method for treating a disorder selected from the group consisting of an inflammatory disorder, oxidative stress related disorder, metabolic disorder, disorder related to bone mineral density, and cancer comprising the step of administering to an individual having same a composition comprising an ingredient containing chicoric acid and/or derivatives thereof, and a lactic acid bacterium capable of hydrolysing chicoric acid and/or derivatives thereof to generate tartaric and/or caffeic acid.

11. Method for hydrolysing chicoric acid and/or derivatives thereof to generate tartaric and/or caffeic acid in a composition comprising using lactic acid bacterium.

12. Method in accordance with claim 11, wherein the lactic acid bacterium is a probiotic lactic acid bacterium having an esterase activity.

13. Method in accordance with claim 10, wherein the composition is a food composition.

14. A kit for preparing a food product or food supplement or beverage, comprising at least two parts comprising:
a first part comprising a chicoric acid and/or derivatives thereof containing a food ingredient; and
a second part comprising a lactic acid bacterium capable of hydrolysing chicoric acid and/or derivatives thereof to generate tartaric and/or caffeic acid.

15. The kit of claim 14 wherein the lactic acid bacterium is a probiotic lactic acid bacterium having an esterase activity.

16. A method for enhancing antioxidant capacity in a human or animal comprising the steps of administering to the human or animal a composition comprising an ingredient containing chicoric acid and/or derivatives thereof, and a lactic acid bacterium capable of hydrolysing chicoric acid and/or derivatives thereof to generate tartaric and/or caffeic acid.
